(19) United States
(12) Patent Application Publication
(10) Pub. No.: US 2006/0058869 A1
(43) Pub. Date: Mar. 16, 2006

(54) Coiled ladder stent

(75) Inventors: Jonathan M. Olson, San Jose, CA (US); Peter E. Rosario, Fremont, CA (US); Guruswami Ravichandran, Arcadia, CA (US); Marshall Tsuruda, San Jose, CA (US)

Correspondence Address: HAYNES BEFFEL & WOLFELD LLP, P O BOX 366, HALF MOON BAY, CA, 94019, US

(73) Assignee: VASCULAR ARCHITECTS, INC., a Delaware Corporation, San Jose, CA (US)

(21) Appl. No.: 10/941,057

(22) Filed: Sep. 14, 2004

(51) Int. Cl. A61F 2/06

(52) U.S. Cl. 623/1.15

(57) ABSTRACT

A coiled ladder stent comprises first and second rails and rungs connecting the rails. At least one rung has lower strength portion or is an enhanced flexibility rung, or both. The lower strength portion is positioned at a reduced strength fracture location along the rung so to promote any fracture of the ladder stent at the fracture location along the rung to help prevent fracture of a rail. One way to make the lower strength portion is to make the cross-sectional area of the lower strength portion less than the average cross-sectional area of the rung. The enhanced flexibility aspect helps to accommodate relative movement between the first and second rails. Doing so helps to prevent fracture of the rails and rungs. In some embodiments of the invention the enhanced flexibility rung comprises one or more nonlinear, such as generally S-shaped or generally U-shaped, portions.

### CROSS-REFERENCE TO OTHER APPLICATIONS

None.

### FEDERALLY SPONSORED RESEARCH OR DEVELOPMENT

None.

### BACKGROUND OF THE INVENTION

The present invention is directed to the rungs of a ladder stent.

Stents, covered stents and other endoluminal prostheses are often useful for placement in various-hollow body structures, such as blood vessels, including coronary arteries, iliac arteries and femoro-popiliteal arteries, the ureter, urethra, bronchus, biliary tract, gastrointestinal tract and the like, for the treatment of conditions which may benefit from the introduction of a reinforcing or protective structure and/or the introduction of a therapeutic agent within the body lumen. The prostheses will typically be placed endoluminally. As used herein, “endoluminally” will mean placement by percutaneous or cutdown procedures, wherein the prosthesis is transluminally advanced through the body lumen from a remote location to a target site in the lumen. In vascular procedures, the prostheses will typically be introduced “endovascularly” using a catheter over a guidewire under fluoroscopic, or other imaging system, guidance. The catheters and guidewires may be introduced through conventional access sites to the vascular system, such as through the femoral artery, or brachial and subclavian arteries, for access to the target site.

An endoluminal prosthesis typically comprises at least one radially expansible, usually cylindrical, body segment. By “radially expansible,” it is meant that the body segment can be converted from a small diameter configuration (used for endoluminal placement) to a radially expanded, usually cylindrical, configuration, which is achieved when the prosthesis is implanted at the desired target site. The prosthesis may be non-resilient, e.g., malleable, thus requiring the application of an internal force to expand it at the target site. Typically, the expansive force can be provided by a balloon catheter, such as an angioplasty balloon for vascular procedures. Alternatively, the prosthesis can be self-expanding. Such self-expanding structures may be provided by a temperature-sensitive superelastic material, such as Nitinol, which naturally assumes a radially expanded condition once an appropriate temperature has been reached. The appropriate temperature can be, for example, a temperature slightly below normal body temperature; if the appropriate temperature is above normal body temperature, some method of heating the structure must be used. Another type of self-expanding structure uses resilient material, such as a stainless steel or superelastic alloy, and forming the body segment so that it possesses its desired, radially-expanded diameter when it is unconstrained, e.g., released from radially constraining forces of a sheath. To remain anchored in the body lumen, the prosthesis will remain partially constrained by the lumen. The self-expanding prosthesis can be delivered in its radially constrained configuration, e.g. by placing the prosthesis within a delivery sheath or tube and retracting the sheath at the target site. Such general aspects of construction and delivery modalities are well known in the art.

The dimensions of a typical endoluminal prosthesis will depend on its intended use. Typically, the prosthesis will have a length in the range from 0.5 cm to 25 cm, usually being from about 0.8 cm to 10 cm, for vascular applications. The small (radially collapsed) diameter of cylindrical prostheses will usually be in the range from about 1 mm to 10 mm, more usually being in the range from 1.5 mm to 6 mm for vascular applications. The expanded diameter will usually be in the range from about 2 mm to 50 mm, preferably being in the range from about 3 mm to 15 mm for vascular applications and from about 25 mm to 45 mm for aortic applications.

One type of endoluminal prosthesis includes both a stent component and a covering component. These endoluminal prostheses are often called stent grafts or covered stents. A covered stent is typically introduced using a catheter with both the stent and covering in contracted, reduced-diameter states. Once at the target site, the stent and covering are expanded. After expansion, the catheter is withdrawn from the vessel leaving the covered stent at the target site. Coverings may be made of, for example, PTFE, ePTFE or Dacron® polyester.

Grafts are used within the body for various reasons, such as to repair damaged or diseased portions of blood vessels such as may be caused by injury, disease, or an aneurysm. It has been found effective to introduce pores into the walls of the graft to provide ingrowth of tissue onto the walls of the graft. With larger diameter grafts, woven graft material is often used. In small and large diameter vessels, porous fluoropolymers, such as ePTFE, have been found useful.

Coil-type stents can be wound about the catheter shaft in torqued compression for deployment. The coil-type stent can be maintained in this torqued compression condition by securing the ends of the coil-type stent in position on a catheter shaft. The ends are released by, for example, pulling on wires once at the target site. See, for example, U.S. Pat. Nos. 5,372,600 and 5,476,505. Alternatively, the endoluminal prosthesis can be maintained in its reduced-diameter condition by a sleeve; the sleeve can be selectively retracted to release the prosthesis. A third approach is the most common. A balloon is used to expand the prosthesis at the target site. The stent is typically extended past its elastic limit so that it remains in its expanded state after the balloon is deflated and removed. One balloon expandable stent is the Palmaz-Schatz stent available from the Cordis Division of Johnson & Johnson. Stents are also available from Medtronic AVE of Santa Rosa, Calif. and Guidant Corporation of Indianapolis, Ind. A controlled release catheter assembly, such as disclosed in U.S. Pat. No. 6,238,430 or 6,248,122, may also be used to deploy a coiled prosthesis. See also U.S. Pat. No. 6,572,643.

The following patents may be of interest. U.S. Pat. No. 6,660,032 issued Dec. 9, 2003; U.S. Pat. No. 6,645,237 issued Nov. 11, 2003; U.S. Pat. No. 6,572,648 issued Jun. 3, 2003; U.S. Pat. No. 4,760,849 issued Aug. 2, 1988; and U.S. Pat. No. 4,553,545 issued Nov. 19, 1985.

### BRIEF SUMMARY OF THE INVENTION

Stents and covered stents may be placed in locations, such as bronchus, esophagus, biliary tracts, that subject the stent to a relatively benign mechanical manipulation environment. Other locations, such as femoro-popiliteal arteries, coronary arteries, and sub-clavian arteries/veins, subject a stent to relatively severe mechanical manipulation environments and cause the stent to repeatedly undergo flexion, compression, extension, or torsion, or a combination thereof. For example, during a typical day a stent placed in the superior femoral artery could experience 3000 cycles of combined flexion and compression on top of normal pulsatile fatigue loading.

It has been found that the rails or the rungs, of conventional ladder stents may fail under the severe mechanical manipulation environments. While rung failures generally do not reduce the overall effectiveness of the stent, it is best avoided. The competing demands of flexibility, strength, durability and biocompatibility create significant obstacles to the design of ladder stents.

One feature of the invention is the recognition of the need to design ladder stents so that the rungs and rails are unlikely to fail, but if either of the rungs or rails is to fail, it is the rung, not the rail, that is the preferred failure mode. Another feature of the invention is the recognition of the need to design ladder stents so that if a rung does fail, it fails at a location that reduces or minimizes any negative consequences from the rung failure. Such locations will generally be referred to as fail safe or fracture safe locations.

A first aspect of the present invention is directed to a coiled ladder stent comprising first and second rails and rungs connecting the rails. At least a first rung comprises a lower strength portion. The lower strength portion is positioned at a reduced strength fracture location along the first rung so to promote any fracture of the ladder stent at the fracture location along the rung to help prevent fracture of a rail.

In some embodiments a number of the rungs, and preferably all of the rungs, may include lower strength portions. One way to make the lower strength portion is to make the cross-sectional area of the lower strength portion less than the average cross-sectional area of the rung. The lower strength portion may also be made by for example, using mechanical, chemical or heat-treating techniques to reduce the strength of such portion. The ladder stent may have rails with end portions joined to one another and central portions oriented parallel to one another.

A second aspect of the invention is directed to a coiled ladder stent comprising first and second rails and rungs connecting the rails. At least a first rung is an enhanced flexibility rung that helps to accommodate relative movement between the first and second rails. Doing so helps to prevent fracture of the rails and rungs.

In some embodiments of the invention the enhanced flexibility rung comprises one or more nonlinear portions. The nonlinear portion may include a curved portion, for example, a generally S-shaped portion, a generally U-shaped portion, or a generally V-shaped portion. The enhanced flexibility rung may also include a portion having an opening formed therethrough; the opening may extend along the entire length of the rung.

Various features and advantages of the invention will appear from the following description in which the preferred embodiments have been set forth in detail in conjunction with the accompanying drawings.

### BRIEF DESCRIPTION OF THE DRAWINGS

FIG. 1 illustrates a conventional ladder stent blank;

FIG. 2 illustrates a conventional coiled ladder stent;

FIGS. 3-12 are enlarged views of sections of several embodiments of ladder stents made according to the invention;

FIGS. 3-5 illustrate three embodiments of the invention in which at least one rung has a lower strength portion at a centrally located, reduced strength fracture location along the rung;

FIGS. 6-10 illustrate five different embodiments of the invention in which at least one rung is an enhanced flexibility rung, with the enhanced flexibility rungs of FIGS. 6-9 having one or more nonlinear portions and with the enhanced flexibility rung of FIG. 10 having an opening extending along the length of the rung;

FIGS. 11-12 illustrate further embodiments of the invention in which the rung is an enhanced flexibility rung with a lower strength portion.

### DETAILED DESCRIPTION OF THE INVENTION

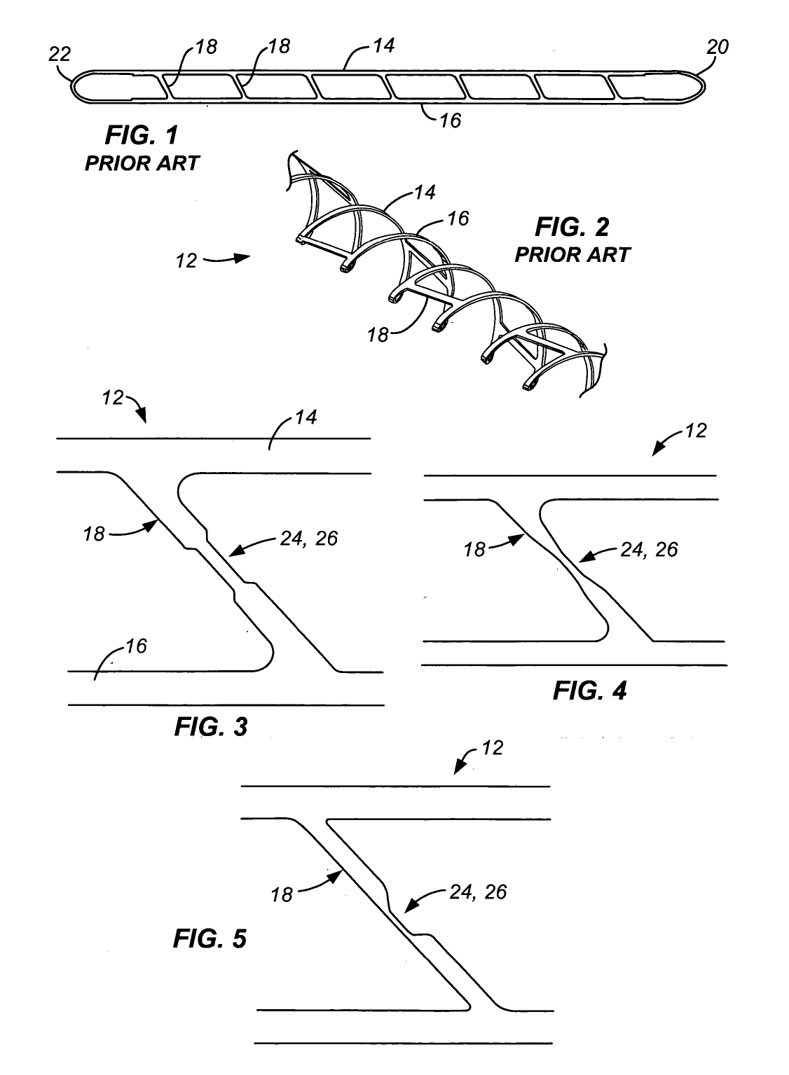

FIG. 1 illustrates a conventional ladder stent blank 10 from which a conventional ladder stent 12, shown in FIG. 2, is formed. Each of ladder stent hank 10 and ladder stent 12 has rails 14, 16 and rungs 18 connecting the rails. Also, rails 14, 16 join together at their distal ends 20, 22. Stent 12 is chemically photoetched from a flat sheet of material (dimensions appropriate for desired final stent size), heat shape set on a mandrel to a helical/coiled form and then surface treated to final product dimension and form.

Rungs 18 are preferably oriented at an acute angle to rails 14 so that when ladder stent 12 is wound down onto a delivery catheter, or other delivery device, rungs 18 are oriented generally parallel to the delivery device axis. This provides a smoother appearance and thus aids passage through the vasculature and to the target site. This feature is further discussed in U.S. Pat. No. 6,660,032.

FIGS. 3-12 are enlarged views of sections of several embodiments of ladder stents made according to the invention with like reference numerals referring to like elements.

In FIGS. 3, 4 and 5, rungs 18 have lower strength portions 24 at reduced strength fracture locations 26. In these embodiments the strength of rung 18 has been reduced by creating a necked-down, reduced cross-sectional area at location 26. Other methods for reducing the strength of rungs 18 at locations 26, such as by the application of heat or chemicals or by mechanically manipulating rungs 18 at locations 26, and also be used.

The fracture location 26 is preferably located along rung 18 so to help reduce any negative consequences of any fracture of the rung. That is, if a rung is fractured at a central fracture location, the lengths of the fractured rung segments will be substantially (that is about 40-60%) shorter than a rung segment that has failed adjacent to a rail 14, 16.

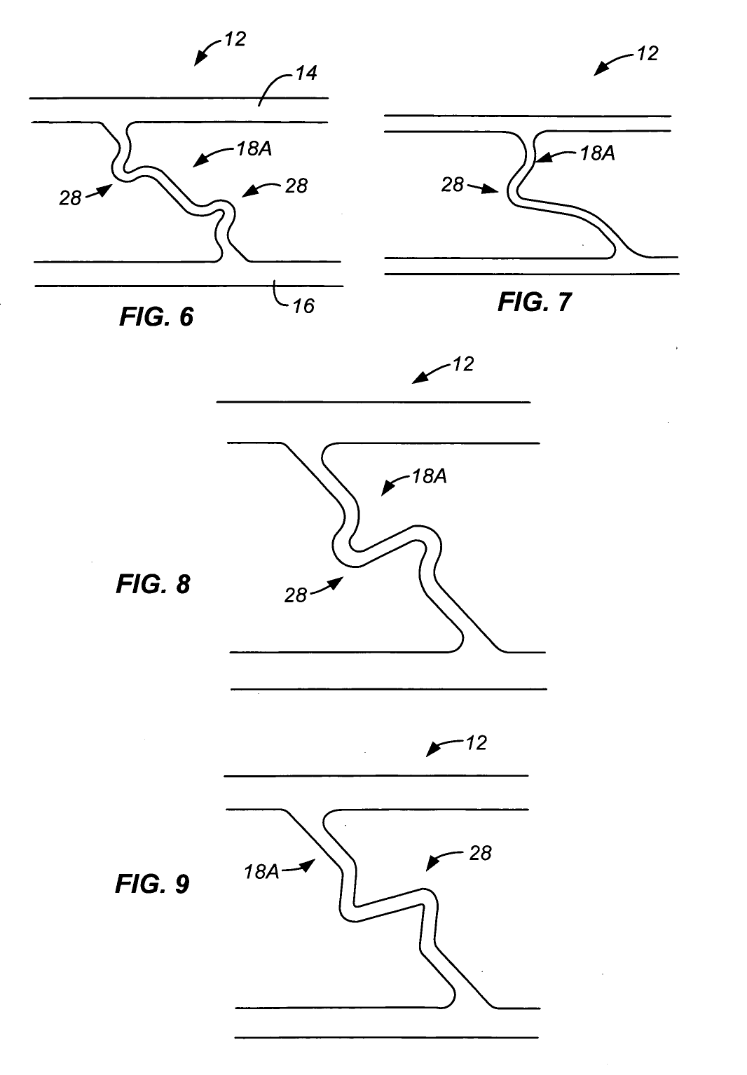

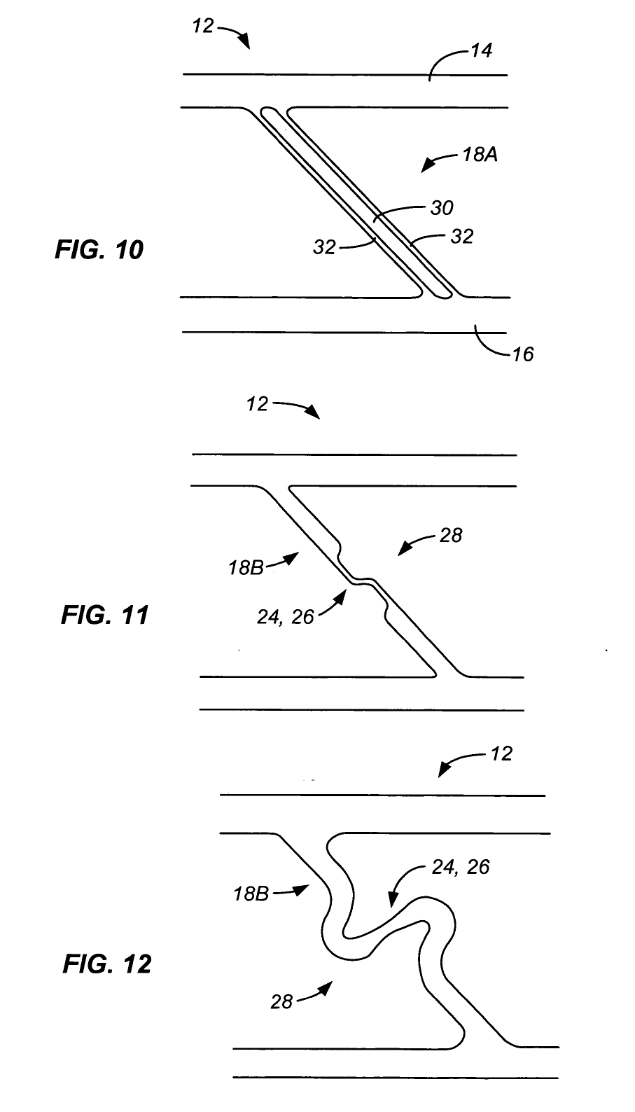

FIGS. 6-10 illustrate sections of five additional embodiments of ladder stents 12 made according to the invention. In these embodiments the rungs are enhanced flexibility rungs 18A to permit relative movement between rails 14, 16 relative to one another when rungs 18A are placed in compression, extension, flexion or torsion, or a combination thereof. The enhanced flexibility of stents is most desirable in these situations because this endows the stents with the ability to conform to the tortuous vessels, thus providing proper apposition to the vessel wall. In addition, flexibility of the stent prevents them from being fixated at discontinuities resulting in stress concentrations and hence failure. The discontinuities may be due to changes in geometry of the vessel or disease such as arterioscleroses or aneurysm. Enhanced flexibility rung means the rung is configured to be more flexible than a similar rung having the same cross-sectional area. The enhanced flexibility rungs 18A of FIGS. 6-9 comprise nonlinear, enhanced flexibility portions 28. That is, portions 28 are not straight as compared to the rungs 18 of FIG. 1. The nonlinear shapes may be characterized as, for example, generally S-shaped (FIGS. 8 and 9), generally U-shaped (FIGS. 6 and 8), generally V-shaped (FIGS. 7 and 9). Other nonlinear shapes may also be used. Nonlinear portion 28 may extend substantially the entire length of rung 18A, as in FIG. 7, or just along a portion, such as about half, of the length of the rung. In the embodiment of FIG. 10, rung 18A has an opening 30 extending along the entire length of the rung. Thus, rung 18A of FIG. 10 is essentially a bifurcated rung having two parallel portions 32. The total cross-sectional area of portions 32 is typically at least as great as the cross-sectional area of the corresponding rungs of FIGS. 6-9 but because of its bifurcation it is more flexible, less stiff, than such rungs.

FIGS. 11 and 12 illustrate two embodiments in which rungs 18 have both the lower strength portions 24 of FIGS. 3-5 and the enhanced flexibility portions 28 of the FIGS. 6-9. Ladder stents embodying a combined fail-safe and flexibility rung utilize the enhancements of both modifications. These ladder stents would have the advantage of flexibility/conformability when placed in tortuous vessels but also have the protection of controlled failure points. These dual advantages would allow for the stent composition to remain intact and allow the stent function to continue even with a rung failure.

Stent 12 is, in one preferred embodiment, made of Nitinol; other conventional or non-conventional materials, such as stainless steel, cobalt alloys, tantalum or polymers, can also be used. Stents 12 may be used as a plain, uncovered stent, or it may have a therapeutic, diagnostic or other agent applied to it or incorporated into it, or it may be used as part of a covered stent and enclosed within a graft material, as in, for example, U.S. Pat. No. 4,553,545 or 6,238,430.

Other modification and variation can be made to the disclosed embodiments without departing from the subject of the invention as defined in following claims.

Any and all patents, patent applications and printed publications referred to above are incorporated by reference.

What is claimed is:

1. A coiled ladder stent comprising:
first and second rails;
rungs connecting the rails; and
at least a first rung comprising a lower strength portion, said lower strength portion positioned at a reduced strength fracture location along the first rung so to promote any fracture of the ladder stent at the fracture location to help prevent fracture of a rail.

2. The ladder stent according to claim 1 wherein a plurality of said rungs comprises lower strength portions.

3. The ladder stent according to claim 1 wherein said fracture location is centrally located along said first rung so to help reduce any negative consequences of any fracture of the first rung.

4. The ladder stent according to claim 1 wherein said first rung has an average cross-sectional area and the lower strength portion has a reduced cross-sectional area relative to the average cross-sectional area.

5. The ladder stent according to claim 1 wherein the rails have end portions and have central portions oriented parallel to one another.

6. The ladder stent according to claim 1 wherein the rungs are oriented at acute angles to the rails.

7. A coiled ladder stent comprising:
first and second rails;
rungs connecting the rails;
each of a plurality of said rungs comprising a lower strength portion, said lower strength portion positioned at a reduced strength fracture location along each of the plurality of said rungs so to promote any fracture of the ladder stent at the fracture location to help prevent fracture of a rail;
said fracture location being centrally located along each of the plurality of said rungs so to help reduce any negative consequences of any fracture any of the plurality of said rungs; and
each of the plurality of said rungs having an average cross-sectional area and the lower strength portion of each of the plurality of said rungs having a reduced cross-sectional area relative to the average cross-sectional area.

8. A coiled ladder stent comprising:
first and second rails;
rungs connecting the rails; and
at least a first rung comprising an enhanced flexibility rung, the enhanced flexibility rung helping to accommodate relative movement between the first and second rails.

9. The ladder stent according to claim 8 morning wherein the enhanced flexibility rung comprises a plurality of nonlinear portions.

10. The ladder stent according to claim 8 wherein the enhanced flexibility rung comprises a nonlinear portion.

11. The ladder stent according to claim 10 wherein the nonlinear portion comprises a curved portion.

12. The ladder stent according to claim 10 wherein the nonlinear portion comprises a generally S-shaped portion.

13. The ladder stent according to claim 10 wherein the nonlinear portion comprises a generally U-shaped portion.

14. The ladder stent according to claim 10 wherein the nonlinear portion comprises a generally V-shaped portion.

15. The ladder stent according to claim 8 wherein the enhanced flexibility rung comprises a portion having an opening formed therethrough.

16. The ladder stent according to claim 15 wherein first rung has a length and the opening extends along the length.

17. The ladder stent according to claim 8 wherein the rails have end portions and have central portions oriented parallel to one another.

18. The ladder stent according to claim 8 wherein the rungs are oriented at acute angles to the rails.

19. A coiled ladder stent comprising:
first and second rails;
rungs connecting the rails;
each of at least some of said rungs comprising an enhanced flexibility rung, the enhanced flexibility rung helping to accommodate relative movement between the first and second rails;
each of a plurality of said rungs comprising a lower strength portion, said lower strength portion positioned at a reduced strength fracture location along each of the plurality of said rungs so to promote any fracture of the ladder stent at the fracture location to help prevent fracture of a rail;
said fracture location being centrally located along each of the plurality of said rungs so to help reduce any negative consequences of any fracture any of the plurality of said rungs; and
each of the plurality of said rungs having an average cross-sectional area and the lower strength portion of each of the plurality of said rungs having a reduced cross-sectional area relative to the average cross-sectional area.
